SEQUENCE LISTING (41 sequences)

SEQ ID NO: 1 - protein, 15 aa, Homo sapiens
YADKPETTKEQLGEF

SEQ ID NO: 2 - protein, 14 aa, Homo sapiens
ADKPETTKEQLGEF

SEQ ID NO: 3 - protein, 10 aa, Homo sapiens
  misc_feature (1)..(1): Oxidation of methionine
MGKVVNPTQK

SEQ ID NO: 4 - protein, 23 aa, Homo sapiens
EDPQGDAAQKTDTSHHDQDHPTF

SEQ ID NO: 5 - protein, 13 aa, Homo sapiens
YVEKGTQGKIVDL

SEQ ID NO: 6 - protein, 12 aa, Homo sapiens
LMIEQNTKSPLF

SEQ ID NO: 7 - protein, 10 aa, Homo sapiens
MGKVVNPTQK

SEQ ID NO: 8 - protein, 9 aa, Homo sapiens
YVVHTNYDE

SEQ ID NO: 9 - protein, 7 aa, Homo sapiens
YGRAPQL

SEQ ID NO: 10 - protein, 21 aa, Homo sapiens
  MISC_FEATURE (13)..(13): Hydroxylation
NGDDGEAGKPGRPGERGPPGP

SEQ ID NO: 11 - protein, 28 aa, Homo sapiens
  MISC_FEATURE (6)..(6): Hydroxylation
GANGAPGNDGAKGDAGAPGAPGSQGAPG

SEQ ID NO: 12 - protein, 14 aa, Homo sapiens
  MISC_FEATURE (2)..(2): Hydroxylation
SPGSPGPDGKTGPP

SEQ ID NO: 13 - protein, 26 aa, Homo sapiens
  MISC_FEATURE (5)..(5): Hydroxylation
ADGQPGAKGEPGDAGAKGDAGPPGPA

SEQ ID NO: 14 - protein, 24 aa, Homo sapiens
  MISC_FEATURE (12)..(12): Hydroxylation
GKNGDDGEAGKPGRPGERGPPGPQ

SEQ ID NO: 15 - protein, 11 aa, Homo sapiens
  MISC_FEATURE (2)..(2): Hydroxylation
SPGPDGKTGPP

SEQ ID NO: 16 - protein, 26 aa, Homo sapiens
  MISC_FEATURE (4)..(4): Hydroxylation
NGAPGNDGAKGDAGAPGAPGSQGAPG

SEQ ID NO: 17 - protein, 22 aa, Homo sapiens
  MISC_FEATURE (4)..(4): Hydroxylation
EGSPGRDGSPGAKGDRGETGPA

SEQ ID NO: 18 - protein, 25 aa, Homo sapiens
  MISC_FEATURE (11)..(11): Hydroxylation
ADGQPGAKGEPGDAGAKGDAGPPGP

SEQ ID NO: 19 - protein, 17 aa, Homo sapiens
  MISC_FEATURE (3)..(3): Hydroxylation
GSPGSPGPDGKTGPPGP

SEQ ID NO: 20 - protein, 26 aa, Homo sapiens
  MISC_FEATURE (11)..(11): Hydroxylation
ADGQPGAKGEPGDAGAKGDAGPPGPA

SEQ ID NO: 21 - protein, 30 aa, Homo sapiens
  MISC_FEATURE (8)..(8): Hydroxylation
ERGEAGIPGVPGAKGEDGKDGSPGEPGANG

SEQ ID NO: 22 - protein, 28 aa, Homo sapiens
  MISC_FEATURE (3)..(3): Hydroxylation
GAPGQNGEPGGKGERGAPGEKGEGGPPG

SEQ ID NO: 23 - protein, 21 aa, Homo sapiens
  MISC_FEATURE (2)..(2): Hydroxylation
APGAPGGKGDAGAPGERGPPG

SEQ ID NO: 24 - protein, 19 aa, Homo sapiens
  MISC_FEATURE (5)..(5): Hydroxylation
NDGAPGKNGERGGPGGPGP

SEQ ID NO: 25 - protein, 28 aa, Homo sapiens
  MISC_FEATURE (3)..(3): Hydroxylation
GAPGQNGEPGGKGERGAPGEKGEGGPPG

SEQ ID NO: 26 - protein, 30 aa, Homo sapiens; identical to SEQ ID NO: 21
  MISC_FEATURE (8)..(8): Hydroxylation

SEQ ID NO: 27 - protein, 20 aa, Homo sapiens
  MISC_FEATURE (6)..(6): Hydroxylation
GNDGAPGKNGERGGPGGPGP

SEQ ID NO: 28 - protein, 18 aa, Homo sapiens
  MISC_FEATURE (4)..(4): Hydroxylation
DGAPGKNGERGGPGGPGP

SEQ ID NO: 29 - protein, 24 aa, Homo sapiens
  MISC_FEATURE (8)..(8): Hydroxylation
DAGAPGAPGGKGDAGAPGERGPPG

SEQ ID NO: 30 - protein, 24 aa, Homo sapiens
  MISC_FEATURE (5)..(5): Hydroxylation
DAGAPGAPGGKGDAGAPGERGPPG

SEQ ID NO: 31 - protein, 23 aa, Homo sapiens
  MISC_FEATURE (4)..(4): Hydroxylation
NGEPGGKGERGAPGEKGEGGPPG

SEQ ID NO: 32 - protein, 30 aa, Homo sapiens
  MISC_FEATURE (16)..(16): Hydroxylation
KNGETGPQGPPGPTGPGGDKGDTGPPGPQG

SEQ ID NO: 33 - protein, 18 aa, Homo sapiens
DEAGSEADHEGTHSTKRG

SEQ ID NO: 34 - protein, 17 aa, Homo sapiens
DEAGSEADHEGTHSTKR

SEQ ID NO: 35 - protein, 9 aa, Homo sapiens
  MISC_FEATURE (2)..(2): Hydroxylation
LPDGSAQGT

SEQ ID NO: 36 - protein, 11 aa, Homo sapiens
EKQLYNKYPDA

SEQ ID NO: 37 - protein, 13 aa, Homo sapiens
DSRGKDSYETSQL

SEQ ID NO: 38 - protein, 11 aa, Homo sapiens
YSRTQTPRAEL

SEQ ID NO: 39 - protein, 10 aa, Homo sapiens
YRSPHWGSTY

SEQ ID NO: 40 - protein, 9 aa, Homo sapiens
WRQVEGMED

SEQ ID NO: 41 - protein, 9 aa, Homo sapiens
  MISC_FEATURE (7)..(7): Oxidation
WRQVEGMED

(19) United States
(12) Patent Application Publication
(10) Pub. No.: US 2008/0227117 A1
(43) Pub. Date: Sep. 18, 2008

(54) Peptides as Biomarkers of Copd

(75) Inventors: Thomas Fehniger, Lund (SE); Claes Lindberg, Lund (SE); Gyorgy Marko-Varga, Lund (SE); Per Broberg, Dalby (SE)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/912,728

(22) PCT Filed: Apr. 27, 2006
(86) PCT No.: PCT/SE06/00507

§ 371 Date: Oct. 26, 2007

(30) Foreign Application Priority Data
Apr. 29, 2005 (GB) 0508863.8

(51) Int. Cl. G01N 33/53; C07K 14/00; C12N 9/00; G01N 33/00; G01N 33/566; C12Q 1/00; C12Q 1/02

(52) U.S. Cl. 435/7.8; 530/395; 530/386; 530/356; 530/399; 530/382; 435/183; 436/86; 435/4; 435/29; 436/501

(57) ABSTRACT

The present invention relates to the identification of biomarkers for the disease condition COPD. The uses of such biomarkers in diagnosis and therapy and a novel method for their identification is are also described.

The present invention relates to the identification of biomarkers for the disease conditions related to Chronic Obstructive Pulmonary Disease (COPD). The uses of such biomarkers in diagnosis and therapy and a novel method for their identification are also described.

### INTRODUCTION

Various biological markers, known as biomarkers, have been identified and studied through the application of biochemistry and molecular biology to medical and toxicological states. A biomarker can be described as “a characteristic that is objectively measured and evaluated as an indicator of normal biologic processes, pathogenic processes, or pharmacological responses to a therapeutic intervention”. A biomarker is any identifiable and measurable indicator associated with a particular condition or disease where there is a correlation between the presence or level of the biomarker and some aspect of the condition or disease (including the presence of, the level or changing level of, the type of, the stage of, the susceptibility to the condition or disease, or the responsiveness to a drug used for treating the condition or disease). The correlation may be qualitative, quantitative, or both qualitative and quantitative. Typically a biomarker is a compound, compound fragment or group of compounds. Such compounds may be any compounds found in or produced by an organism, including proteins (and peptides), nucleic acids and other compounds.

Biomarkers may have a predictive power, and as such may be used to predict or detect the presence, level, type or stage of particular conditions or diseases (including the presence or level of particular microorganisms or toxins), the susceptibility (including genetic susceptibility) to particular conditions or diseases, or the response to particular treatments (including drug treatments). It is thought that biomarkers will play an increasingly important role in the future of drug discovery and development, by improving the efficiency of research and development programmes. Biomarkers can be used as diagnostic agents, monitors of disease progression, monitors of treatment and predictors of clinical outcome. For example, various biomarker research projects are attempting to identify markers of specific cancers and of specific cardiovascular and immunological diseases.

Proteomics (including peptidomics) technologies have been developed to analyse proteins and peptides. These technologies are applied in a high-throughput mode, generating an enormous amount of data that is analysed using computer systems. Proteins from a biological sample are isolated and separated at a high resolution, for example by chromatographic separations. The set of proteins is then characterised using qualitative and quantitative techniques such as mass spectrometry. The result is a protein (or peptide) fingerprint (a constant, reproducible set of proteins or peptides). Selected proteins/peptides or groups of proteins/peptides may be analysed further to generate protein/peptide profiles. Proteormics is now viewed as the large-scale analysis of the function of genes and is becoming a central field in functional genomics.

COPD is a disease condition, which has chronic cigarette smoking as the principle determinant risk factor. Other diseases and conditions are also associated with chronic cigarette smoking history including cancer, hypertension, emphysema, chronic bronchitis, stroke, and coronary heart disease. The pathophysiology of COPD is characterized by pulmonary inflammation, increased mucus production, narrowing of airways, hyperinflation, and destruction of the alveolar walls leading to emphysema. One key event in the onset of COPD is a progressive loss of lung tissue including matrix components such as elastin and collagen, which are important structural components of alveolar walls and pulmonary vessels. Increased levels of elastin degradation products (elastin peptides and desmosine) in BALF (Bronchoalveolar fluid), blood and urine have been reported in smokers and COPD patients (1,2). These products serve as markers of the lung elastolytic activity in COPD. Elastolytic enzymes such as neutrophil elastase, MMP-2,-9,-7,-12, cathepsin G, S, L and K, proteinase 3, as well as collagenolytic proteases such as MMP-1 and -8 released from cells during the inflammatory response are presumed to be important in this degradative process (3). Other diseases or conditions, which elaborate proteases and protease activities, include for example cancer, interstitial lung disease, alpha-1-anti-trypsin deficiency, emphysema, bronchiectasis, and some infections.

IL-8 and LTB4 are important local neutrophil chemo-attractants in the airways and TNF-α, IL-6, IL-1β, GRO-α and ENA-78 inflammatory mediators crucial for airway inflammation and leading development of lung emphysema (4). In COPD local inflammation in the lung may be manifested as a general systemic inflammation, which is a risk factor for complications like atherosclerosis, cachexia, anorexia and osteoporosis and all of these complications are commonly observed in patients with COPD. Significant increase in plasma biomarkers of inflammation such as CRP, fibrinogen, TNF-α and leukocyte number has been observed in a number of studies of stable COPD patients. It has also been observed that about half of the COPD patients die from systemic cardiovascular causes (5).

Development of specific biochemical markers would enable identification of individuals with highly elevated degradation/inflammation status. It is possible that a marker of lung tissue destruction such as emphysema development would provide a new basis for classification of COPD patients, and which may offer a significant clinical potential for selecting treatments, optimised to halt emphysema development in the individual patient.

Recent developments in proteomic technologies today allow the profiling of low molecular weight peptides in biological fluids. Technology platforms based on enrichment of peptides on surfaces followed by mass spectrometry (SELDI) (6) or peptide separation by LC and mass spectrometric detection (7) allow the detection of thousands of peptides in a single sample. Combined with the proper statistical analyses these techniques may potentially be used to identify biomarkers indicative of disease states.

There remains a need in the art for a sensitive and reproducible method for identifying COPD associated markers in biological fluids, in particular urine. Moreover, the identification of biomarkers of COPD would facilitate the diagnosis and/or treatment of this and related disorders.

### SUMMARY OF THE INVENTION

The present inventors sought to identify COPD disease associated and naturally occurring peptide biomarkers within the biological fluids of subjects. In order to solve the recognised technical problems encountered in recent studies using SELDI (8), such as reproducibility and sensitivity, the present inventors have modified an LC-MALDI based platform for separating and measuring peptides. This platform provides the capacity and sensitivity required for the detection and quantification of multiple peptides in biological samples, in particular urinary samples.

Thus, in one aspect the present invention provides a method for the detection of one or more biomarkers of the disease COPD which method comprises the steps of:

(a) Obtaining a sample of biological fluid selected from the group consisting of: blood, urine, plasma, sputum, serum, saliva, cerebral-spinal fluid, sweat or tissue extracts.

(b) Subjecting the material according to step (a) to one-dimensional or multi-dimensional liquid chromatography (nD-LC);

(c) Subjecting the material according to step (b) to mass spectrometry, and

(d) Identifying the peptides of interest within the biological sample treated as described in steps (b) and (c) from the output from the mass-spectrometry step (b).

The method described herein is suitable for the detection and/or separation of one or more peptides from any suitable biological fluid. Suitable biological fluids include but are not limited to any of the following: lung tissue, urine; sputum; tears, blood; serum; plasma, synovial fluid; cerebral spinal fluid; ascites fluid and sweat. Advantageously, the method of the invention is most suited for the identification and/or separation of one or more peptides of interest from urinary fluid.

The method of the invention is for the detection and/or quantitation of one or more peptides, in either natural form or as modified products resulting from chemical or physical treatments of the biosample, and which are indicative of the disease COPD. Advantageously peptides identified and/or quantitated using the method described herein include those in the group consisting of the following in singular form or in any combination: zinc-alpha-2-glycoprotein, alpha-i-antitrypsin, collagen type III, prostaglandin-H2 D isomerase, collagen type I, alpha-1-microglobulin, fibroblast growth factor, osteopontin, alpha-1 acid glycoprotein 2, fibrinogen alpha-E chain and any other peptides or groups thereof recited herein. Those slidlled in the art will appreciate that this list is not intended to be exhaustive.

According to the method of the invention described herein, preferably more than one peptide of interest is detected or quantitated from a given biological fluid. Preferably, more than 2, 3, 4, 5, 8, 10, 12, 15, 18, 20, 25, 28, 30.35, 38, 40, 45, 50, 60, 80 or 100 or more peptides are detected and/or quantitated from one given biological sample using the method of the invention.

In a further aspect the present invention describes a COPD biomarker, wherein that biomarker is any peptide/fragment or any modification of any peptide component of the protein in the group consisting of but not limited to the following: zinc-alpha-2-glycoprotein, alpha-1-antitrypsin, collagen type III, prostaglandin-H2 D isomerase, collagen type I, alpha-1-microglobulin, fibroblast growth factor, osteopontin, alpha-1 acid glycoprotein 2, fibrinogen alpha-E chain and any other peptides or groups thereof recited herein.

Preferably a COPD biomarker according to the invention is any one or more of those peptides provided in SEQ ID Nos 1-41, shown in Table 1 herein.

TABLE 1

| SEQ | Peptide | | | Position of | |
|---|---|---|---|---|---|
| ID NO. | Mr | Peptide sequence | Modification | modification | Precursor protein |
| 1 | 1754.8 | YADKPETTKEQLGEF | | | Alpha-1-acid |
| | | | | | glycoprotein 2 |
| 2 | 1591.8 | ADKPETTKEQLGEF | | | Alpha-1-acid |
| | | | | | glycoprotein 2 |
| 3 | 1116.6 | MGKVVNPTQK | Oxidation | M1 | Alpha-1- |
| | | | | | antitrypsin |
| 4 | 2576.1 | EDPQGDAAQKTDTSH | | | Alpha-1- |
| | | HDQDHPTF | | | antitrypsin |
| 5 | 1448.8 | YVEKGTQGKIVDL | | | Alpha-1- |
| | | | | | antitrypsin |
| 6 | 1419.7 | LMIEQNTKSPLF | | | Alpha-1- |
| | | | | | antitrypsin |
| 7 | 1100.6 | MGKVVNPTQK | | | Alpha-1- |
| | | | | | antitrypsin |
| 8 | 1138.5 | YVVHTNYDE | | | Alpha-1- |
| | | | | | microglobulin |
| 9 | 803.4 | YGRAPQL | | | Alpha-1- |
| | | | | | microglobulin |
| 10 | 2047.9 | NGDDGEAGKPGRPGE | 2 Hydroxylations | P13, P18 | Collagen type I |
| | | RGPPGP | | | |
| 11 | 2339.0 | GANGAPGNDGAKGDA | 4 Hydroxylations | P6, P18, P21, | Collagen type I |
| | | GAPGAPGSQGAPG | | P27 | |
| 12 | 1297.6 | SPGSPGPDGKTGPP | 3 Hydroxylations | P2, P5, P14 | Collagen type I |
| 13 | 2292.0 | ADGQPGAKGEPGDAG | 3 Hydroxylations | P5, P11, P23 | Collagen type I |
| | | AKGDAGPPGPA | | | |
| 14 | 2377.1 | GKNGDDGEAGKPGRP | 3 Hydroxylations | P12, P15, | Collagen type I |
| | | GERGPPGPQ | | P20 | |
| 15 | 1040.5 | SPGPDGKTGPP | 2 Hydroxylations | P2, P10 | Collagen type I |
| 16 | 2211.0 | NGAPGNDGAKGDAGA | 5 Hydroxylations | P4, P16, P19, | Collagen type I |
| | | PGAPGSQGAPG | | P25, K10 | |
| 17 | 2085.9 | EGSPGRDGSPGAKGDR | 2 Hydroxylations | P4, P10 | Collagen type I |
| | | GETGPA | | | |
| 18 | 2205.0 | ADGQPGAKGEPGDAG | 2 Hydroxylations | P11, P23 | Collagen type I |
| | | AKGDAGPPGP | | | |
| 19 | 1508.7 | GSPGSPGPDGKTGPPGP | 3 Hydroxylations | P3, P6, P17 | Collagen type I |
| 20 | 2276.0 | ADGQPGAKGEPGDAG | 2 Hydroxylations | P11, P23 | Collagen type I |
| | | AKGDAGPPGPA | | | |
| 21 | 2809.3 | ERGEAGIPGVPGAKGE | 3 Hydroxylations | P8, P11, P26 | Collagen type III |
| | | DGKDGSPGEPGANG | | | |
| 22 | 2564.1 | GAPGQNGEPGGKGER | 4 Hydroxylations | P3, P9, K21, | Collagen type III |
| | | GAPGEKGEGGPPG | | P27 | |
| 23 | 1835.8 | APGAPGGKGDAGAPG | 4 Hydroxylations | P2, P5, P14, | Collagen type III |
| | | ERGPPG | | P20 | |
| 24 | 1737.8 | NDGAPGKNGERGGPG | 3 Hydroxylations | P5, P14, P17 | Collagen type III |
| | | GPGP | | | |
| 25 | 2580.1 | GAPGQNGEPGGKGER | 5 Hydroxylations | P3, P9, K12, | Collagen type III |
| | | GAPGEKGEGGPPG | | K21, P27 | |
| 26 | 2825.3 | ERGEAGIPGVPGAKGE | 4 Hydroxylations | P8, P11, K19, | Collagen type III |
| | | DGKDGSPGEPGANG | | P26 | |
| 27 | 1794.8 | GNDGAPGKNGERGGP | 3 Hydroxylations | P6, P15, P18 | Collagen type III |
| | | GGPGP | | | |
| 28 | 1623.7 | DGAPGKNGERGGPGG | 3 Hydroxylations | P4, P13, P16 | Collagen type III |
| | | PGP | | | |
| 29 | 2063.9 | DAGAPGAPGGKGDAG | 3 Hydroxylations | P8, P17, P22 | Collagen type III |
| | | APGERGPPG | | | |
| 30 | 2078.9 | DAGAPGAPGGKGDAG | 4 Hydroxylations | P5, P8, P17, | Collagen type III |
| | | APGERGPPG | | P23 | |
| 31 | 2138.0 | NGEPGGKGERGAPGE | 3 Hydroxylations | P4, P13, P22 | Collagen type III |
| | | KGEGGPPG | | | |
| 32 | 2743.3 | KNGETGPQGPPGPTGP | 2 Hydroxylations | P16, P25 | Collagen type III |
| | | GGDKGDTGPPGPQG | | | |
| 33 | 1882.8 | DEAGSEADHEGTHSTK | | | Fibrinogen alpha |
| | | RG | | | E-chain |
| 34 | 1825.8 | DEAGSEADHEGTHSTK | | | Fibrinogen alpha |
| | | | | | E-chain |
| 35 | 860.4 | LPDGSAQGT | 1 Hydroxylation | P2 | Human fibroblast |
| | | | | | growth factor |
| 36 | 1367.7 | EKQLYNKYPDA | | | Osteopontin |
| 37 | 1484.7 | DSRGKDSYETSQL | | | Osteopontin |
| 38 | 1320.7 | YSRTQTPRAEL | | | Prostaglandin- |
| | | | | | H2 D-isomerase |
| 39 | 1252.6 | YRSPHWGSTY | | | Prostaglandin- |
| | | | | | H2 D-isomerase |
| 40 | 1164.5 | WRQVEGMED | | | Zinc-alpha-2- |
| | | | | | glycoprotein |
| 41 | 1148.5 | WRQVEGMED | Oxidation | M7 | Zinc-alpha-2- |
| | | | | | glycoprotein |

In a further aspect still, the invention provides a COPD disease profile, wherein that disease profile comprises the identity of two or more COPD biomarkers according to the present invention.

According to the above aspect of the invention, preferably a COPD disease profile comprises one or more of those characteristics in the group consisting of: the identity of two or more COPD biomarkers and quantitative data relating to the number and/or amounts of COPD biomarkers within one or more biological fluids, preferably urine isolated from one or more subjects suffering from COPD.

According to the invention described herein, the term ‘COPD biomarker’ refers to a peptide or protein present and detectable in one or more biological fluids from a patient diagnosed or at risk for developing from COPD. Advantageously, that COPD biomarker is present in urine. A COPD disease profile according to the present invention is generated from one or more biological samples, preferably urine samples derived from a patient diagnosed or at risk for developing COPD. In a preferred embodiment of the above aspect of the invention, the amount (generally measured as a concentration) of one or more COPD biomarkers as herein defined, within the biological fluid of a patient suffering from COPD is not the same when compared with a healthy control, individual as defined herein. The present inventors have often found that the concentration of one or more COPD biomarkers often increases in those patients clinically diagnosed or at risk for developing from COPD in comparison with healthy individual/s with or without histories of smoking. However, a number of COPD biomarkers appear to decrease in concentration in those patients clinically diagnosed or at risk for developing from COPD. One example of such a biomarker is complement C3 precursor protein, which is a key component in complement activation.

According to the above aspect of the invention, the term ‘COPD disease profile’ refers to a representation of a disease state, in this case COPD, such that quantitative and/or sequence information relating to peptides present in one or more biological fluids of one or more patients clinically diagnosed or at risk for developing COPD is presented.

In a further aspect the present invention provides the use of one or more monoclonal antibodies in the group consisting of: those antibodies listed in Table 2 herein, in the identification of one or more COPD biomarkers.

TABLE 2

| Protein id | Immunoassay | Source |
|---|---|---|
| Collagen type I | ELISA, Crosslaps | Nordic |
| peptide | | Biosciences, DK |
| Collagen type III | ELISA, Mab, | Abcam |
| peptide | Polyclonal Abs | Acris antibodies |
| Prostaglandin- | ELISA (reference | Santa Cruz |
| H2D-isomerase | 25), Polyclonal Abs | Cayman |
| Alpha-1- | ELISA | Alpco |
| microglobuline | | Research |
| | | Diagnostics Inc |
| Alpha-1-acid | ELISA, Poly abs | ICL labs |
| glycoprotein | | |
| Alpha-1-antitrypsin | ELISA, Poly abs | ICL labs |
| FGF basic | ELISA | Abcam |
| | | R&D |
| Osteopontin | ELISA | Bioscience |
| | | Technology |
| Fibrinogens | ELISA | Abcam |

Those skilled in the art will appreciate that the identification and/or quantification of COPD biomarkers in one or more biological fluids isolated from one or more subjects suffering from COPD and/or the generation of a ‘COPD disease profile’ as herein described may have many and varied uses.

Importantly, the inventors consider that such information may be used in the diagnosis of COPD. Further, this information may be used in assessing COPD onset, progression and/or the efficacy of one or more treatments of COPD in a subject. Further, it is generally accepted that COPD often results in a progressive loss of lung tissue leading to the progressive development emphysema. Thus, the inventors consider that a COPD disease profile, identified using the method of the invention described herein, may be used in the diagnosis and/or to assess disease progression and/or the effectiveness of one or more palliative or curative emphysema treatment regimens.

Thus in a further aspect, the present invention provides a method for one or more of the following: predicting or diagnosing the onset or occurrence of COPD; assessing predicting or diagnosing the onset or occurrence of emphysema, which method comprises the step of:

(a) Obtaining a sample of biological fluid from a subject to be assessed,

(b) Identifying and/or quantifying the peptides present within that biological fluid and using that data in order to generate a COPD disease profile, and

(c) comparing the disease profile obtained according to step (b) with that of a healthy control subject.

In a further aspect, the present invention provides a method for assessing COPD disease progression and/or assessing the progression of emphysema in a subject which method comprises the step of:

(a) preparing a COPD disease profile at a first time interval, and

(b) comparing that COPD disease profile according to step (a) with a disease profile prepared at a second time interval.

In a further aspect still, the present invention provides a method for assessing the effectiveness of one or more palliative or curative COPD and/or emphysema treatment regimens in a subject which method comprises the step of:

(a) preparing a COPD disease profile at a first time interval, and

(b) comparing that COPD disease profile according to step (a) with a disease profile prepared at a second time interval; wherein the second time interval corresponds to a time subsequent to a COPD and/or emphysema treatment regimen being administered to the subject.

According to the above aspect of the invention, a change in the disease profile between step (a) and step (b) is indicative of the tested treatment regimen having an effect on the subject.

According to the aspects of the invention described herein preferably the one or more COPD biomarkers and/or the COPD profile is generated using the method of the invention described herein.

According to the invention described herein, preferably the COPD disease profile comprises information relating to 2 or more, 3 or more, 4 or more, 5 or more, 8 or more, 10 or more, 12 or more, 15 or more, 18 or more, 20 or more, 25 or more, 28 or more, 30 or more, 35 or more, 38 or more, 40 or more, 45 or more, 50 or more, 60 or more, 80 or more or 100 or more peptides present in one or more biological fluids, preferably urine from one or more patients diagnosed or at risk for developing from COPD.

According to the invention described herein, the term ‘a healthy control subject’ refers to a subject who is not exhibiting one or more signs or symptoms characteristic of COPD; including but not limited to pulmonary inflammation, increased mucus production, chronic bronchitis narrowing of airways, obstruction of the airways, hyperinflation, destruction of alveolar walls and emphysema. According to the present invention, preferably the term ‘a healthy control subject’ does not exhibit clinical signs or symptoms of any one or more diseases named above. A healthy control subject does not clinically present with indications of other known diseases, conditions, or infections.

As eluded to above, the inventors have found that COPD biomarkers often increase in quantity in the biological fluids of diseased patients as compare with normal healthy individuals. Thus, in the case of COPD, at least one biomarker in the group consisting of the following increase in concentration in those patients suffering from COPD: zinc-alpha-2-glycoprotein, alpha-1-antitrypsin, collagen type (III), prostaglandin-H2 D isomerase, collagen alpha 1 (I), alpha-1-microglobulin, fibroblast growth factor, osteopontin, alpha-1 acid glycoprotein 2, fibrinogen alpha-E chain.

Those skilled in the art will appreciate that any fragment of those proteins listed above may be present in the biological fluid, preferably the urine, of a subject diagnosed or at risk for developing COPD. Accordingly, a COPD biomarker as herein defined includes within its scope any peptide fragment of any one or more of the proteins listed above. In a preferred embodiment of the above aspect of the invention a COPD biomarker according to the present invention consists of any of those peptide sequences provided in Table 1 herein.

The inventors have found that within certain protein groups, for example within the collagen type I and collagen type III groups of proteins, samples of COPD patients, in particular within urine, contain more than one peptide derived from these proteins. Moreover, the inventors have found that within a proportion of the protein groups, which contain more than one COPD peptide (COPD biomarker), the concentration of one peptide increases within the biological fluid of the patient whilst another decreases. The inventors have identified the following groups as containing more than one COPD peptide, at least one increasing in concentration and at least one peptide decreases in concentration: collagen type (III), prostaglandin-H2 D isomerase, collagen type I.

### Definitions.

Peptide: The term peptide refers to any fragment of a protein consisting of two or more amino acids joined together by amide bonds. A peptide can be formed by proteolytic cleavage of a protein or from artificial synthesis by linking, enzymatically or non-enzymatically, two or more amino acids together. The amino acids being part of the peptide can be any naturally occurring amino acid, or any modification thereof.

Disease biomarker: According to the invention described herein, the term ‘disease biomarker’ refers to a naturally occurring or modification of a naturally occurring peptide present and detectable in one or more biological fluids from a patient suffering from one or more disease conditions and which is considered to be indicative of that disease or condition. According to the invention described herein, often disease biomarkers identified using the method of the invention will not be detectable in the biological fluid of a healthy control, individual as herein defined. In the case where that ‘disease biomarker’ can be detected within the biological fluid of a healthy individual then the present inventors have found that the concentration or amount of a ‘disease biomarker’ present within the biological fluid of a diseased subject is equal to the concentration or amount of that biomarker in the same type of biological fluid sampled from a healthy (control) subject. That is, the absolute amount of a disease biomarker, in particular a COPD biomarker according to the present invention varies between normal healthy control individuals and those subjects suffering from COPD.

COPD: According to the present invention, the term COPD is a progressive lung disorder/disease characterised by any one or more of the following symptoms: pulmonary inflammation, increased mucus production within the pulmonary tract, narrowing of airways, chronic bronchitis, obstruction of the airways, hyperinflation, and destruction of the alveolar walls leading to emphysema. One key event in the onset if COPD is the progressive loss of lung tissue including matrix components such as elastin and collagen, which are important structural components of the alveolar walls and pulmonary vessels. COPD may also include certain co-morbities of other disease conditions including cancer, systemic inflammation, loss of body mass, hypertension, stroke, certain arthritic conditions, certain infections, and coronary heart disease.

COPD biomarker: According to the invention described herein, the term ‘COPD biomarker’ refers to a ‘disease biomarker’ as defined herein, which is present and detectable in one or more biological fluids from a patient diagnosed or at risk for developing COPD. Advantageously, that COPD biomarker is present in urine. A COPD disease profile according to the present invention is advantageously generated from one or more urine samples derived from a patient suffering from COPD. In a preferred embodiment of the above aspect of the invention, COPD biomarkers identified using the method of the invention are found within the biological fluids of both diseased and non-diseased individuals. However, the inventors have found that the absolute amount (generally measured as a concentration) of one or more COPD biomarkers, within the biological fluid of a patient suffering from COPD is not identical to the amount detected in a so called healthy control individual as defined herein. The present inventors have often found that the relative concentration of one or more COPD biomarkers often increases in those patients suffering from COPD as compared with a healthy individual. However, a number of COPD biomarkers appear to decrease in concentration in those patients suffering from COPD. One example of such a biomarker is complement C3 precursor protein, which is a key component in complement activation.

According to the present invention, a COPD biomarker is any peptide/fragment of any protein in the group consisting of the following: zinc-alpha-2-glycoprotein, alpha-1-antitrypsin, collagen type III, prostaglandin-H2 D isomerase, collagen type I, alpha-1-microglobulin, fibroblast growth factor, osteopontin, alpha-1 acid glycoprotein 2, fibrinogen alpha-E chain and any other peptides or groups thereof recited herein. In a preferred embodiment of the above aspect of the invention a COPD biomarker according to the present invention consists of any of those peptide sequences provided in Table 1 herein.

COPD disease profile: ‘a COPD disease profile’ comprises one or more of those characteristics in the group consisting of: the identity of two or more COPD biomarkers and quantitative data relating to the number and/or amounts of COPD biomarkers within one or more biological fluids, preferably urine isolated from one or more subjects suffering from COPD.

### BRIEF DESCRIPTION OF THE FIGURES

FIG. 1. describes the liquid chromatography instrument set up according to a preferred embodiment of the invention.

### DETAILED DESCRIPTION OF THE INVENTION

Method for the Detection of COPD Biomarkers within Biological Fluid

### (i) Biological Fluid Samples.

Suitable biological fluids for analysis using the method of the invention include any one or more of the following non restrictive examples including: pulmonary biopsies or surgical specimens of tissue, lung tissue, urine; blood; plasma, serum, tears, serum; synovial fluid; cerebral spinal fluid; ascites fluid; lymph; bronchial or nasal washings, lavages, brushings, and biopsies; sputum; and sweat. Those skilled in the art will appreciate that this list is not intended to be exhaustive.

Methods for obtaining samples of biological fluids for use according to the method of the invention are many and varied and will depend upon the nature of the biological material. Suitable methods will be familiar to those skilled in the art.

### (ii) Two-Dimensional Liquid Chromatography.

The present inventors have developed a method, which allows the detection of many peptides of interest from within a sample of biological fluid. Such a method is based upon a combination of two-dimensional liquid chromatography and mass spectrometry and overcomes the problems with the prior art methods (for example SELDI (8)), namely sensitivity and reproducibility.

Two dimensional (2D) liquid chromatography was originally explored as an alternative to ID IPLC separation. This approach uses two orthogonal modes of EPLC (high pressure liquid chromatography) linked in tandem. The 2D chromatography approach permits a significantly improved resolution of proteins in biological fluids as compared with other chromatography techniques. Techniques and apparatus describing the technique of 2D-liquid chromatography will be familiar to those skilled in the art.

### 2D-Chromatography Columns.

Generally, the columns are chosen so that one retention mechanism in the first column (dimension) is vastly different than the retention mechanism in the second column (dimension). Examples typically include a reversed-phase (RP) column in the first dimension followed by a size-exclusion column in the second or an ion-exchange column in the first dimension followed by a RP column in the second dimension.

In a preferred embodiment of the above aspect of the invention, the 2D-chromatography step involves the use of RAM (cation exchange restricted access material), in order to remove high molecular weight material prior to analysing the sample for the presence of one or more peptides of interest. Restricted access cation exchange material is described in Chiap P, et al, Chromatography 2002; 975 (1) 145-55 (51), which is herein incorporated by reference.

The use of restricted-access material (RAM) in the pre-column is an approach, which permits the direct injection of protein-rich samples, such as plasma. A family of restricted access sorbents, namely alkyl diol silica (ADS), belonging to the group of internal surface reversed phase (ISRP) supports, was developed by Boos et al, J. Anal. Chem 352 (1995) 684 and Boos et al, Trends Anal Chem, 18 (1999) 175. In this approach low molecular mass compounds such as peptides can have access to the internal surface of the sorbent, on which either butyl (C₄) Capryloyl (C₈) or stearoyl (C₁₈) moieties are bonded. These compounds are retained mainly by hydrophobic interactions while macromolecules like proteins are excluded and eluted directly from the pre-column. The access restriction is obtained by use of silica particles (25 μm) with an appropriate pore diameter (6 nm). Moreover, the adsorption and denaturation of proteins is prevented by hydrophilic and electroneutral diol groups present on the external surface of the particles.

Those skilled in the art will appreciate that the size and nature of the high molecular weight material present will depend upon the biological fluid analysed. In a preferred embodiment of the above aspect of the invention, high molecular weight material will generally be considered to be greater than 10 kD or more, 20 kD or more, 30 kD or more, 50 kD or more, 100 kD) or more or 150 kD or more.

According to the method of the invention described herein, preferably, the liquid-chromatography step (b) in addition, or alternatively, involves the use of a cation-exchange coluran, preferably a strong-cation exchange column (SCX) for the separation of the one or more peptides of interest.

Those skilled in the art will appreciate that experimental details will be determined by the nature of the biological sample and the size of the peptides of interest. Details of some preferred embodiments of the method of the invention are described in the examples herein.

### 2D-Liquid Chromatogaphy Coupled to Mass Spectrometry.

According to the method of the invention described herein, the output from 2D liquid chromatography is analysed using mass spectrometry.

Methods utilised in the technique of mass-spectrometry will be familiar to those skilled in the art. Details are provided in Aebersold and Mann, Nature 2003 (56).

COPD Biomakers Identified using the Method of the Invention.

In a further aspect the present invention describes a COPD biomarker, wherein that biomarker is any peptide/fragment of any protein in the group consisting of the following: zinc-alpha-2-glycoprotein, alpha-i-antitrypsin, collagen type III, prostaglandin-H2 D isomerase, collagen type I, alpha-λ-microglobulin, fibroblast growth factor, osteopontin, alpha-i acid glycoprotein 2, fibrinogen alpha-E chain and any other peptides or groups thereof recited herein.

In a preferred embodiment of the invention, the COPD biomarker is any one of those peptides listed as SEQ ID NOs 1 to 41 in Table 1

The following section describes the COPD biomarkers based on ranking of differentially expressed peptides and the biological function of the proteins. The proteins are sorted according to the statistical rank of the peptides.

### (i) Protein Descriptions.

### Zinc-Alpha-2-Glycoprotein.

Zinc-alpha -2-glycoprotein (AZGP1) was represented by two peptides, both being upregulated in COPD. The two peptides had the same amino acid sequence, differing in that one of them was oxidized on methionine.

The protein zinc-alpha2-glycoprotein (AZGP1) is a protein of unknown function. AZGP1 is a member of the major histocompatibility complex (MHC) class I family of proteins and is identical in amino acid sequence to a tumour-derived lipid-mobilizing factor associated with cachexia in cancer patients (14). AZGP1 is present in plasma and other body fluids, and its natural function, probably lies in lipid store homeostasis. It has been shown that AZGP1 binds the fluorophore-tagged fatty acid 11-(dansylamino)-undecanoic acid and, by competition, natural fatty acids such as arachidonic, linolenic, eicosapentaenoic, and docosahexaenoic acids (14). Additionally, AZGP1 has been shown to be up-regulated by glucocorticoids in mouse adipocytes, and up in mice with cancer cachexia (15). Furthermore, it has been identified in nasal lavage (NAL) from in smokers, and differentially expressed in NAL in workers challenged by epoxy (16, 17).

### Alpha-1-Antitrypsin.

Alpha-1-antitrypsin was represented by six peptides, all being upregulated in COPD. Two peptides had the same amino acid sequence, differing in that one of them was oxidized on methionine.

Alpha-1-antitrypsin (AAT) is a serine protease inhibitor (SERPI), with a supposed main function to block neutrophil elastase, which is catalytically inactivated by binding to AAT.

AAT is one of the most abundant proteins in plasma (1.3-1.7 mg/ml) and forms the alpha-i band in human whole plasma electrophoresis band pattern. AAT is mainly produced by the liver; half of the AAT is extravascular and display a high turnover rate (half-life of 6 days). AAT production is stimulated by IFN-b, IL-6 and gp130 and since AAT concentrations increase upon infection, it is classified as an acute phase protein (APP).

Humans with genetic AAT deficiency have increased risk of developing lung emphysema and treating these individuals with supplemental AAT has a beneficial effect on lung function (18). As for other acute phase proteins (APPs) naive AAT, or peptides thereof, have various modifying effects on the inflammatory response such as decreasing fMLP-induced PMN adhesion to fibronectin, decreasing Zymosan uptake by macrophages by 70%, decreasing IL-8 release from neutrophils, decreasing production of TNF-α, IL-1, MCP-1, IL-8 and increasing production of IL-10 from human LPS stimulated monocytes (unpublished data).

Cigarette smoke causes an oxidative environment which leads to free radical production causing conformational changes of ATT and dysfunctional variants with alterations in the active site of AAT (acquired AAT deficiency, protease inhibiting function lost). Polymeric forms of AAT are formed and found increased in smokers and COPD patients. In vivo modification of AAT apparently leads to altered turnover of AAT and appearance of novel peptides being formed which display potentially novel pro-inflammatory effects in smokers (unpublished data).

### Collagen Type III.

Collagen type III (CIII) was represented by 10 peptides being upregulated and by 3 peptides being downregulated in COPD.

Sixty percent of the lung proteins consist of collagens among which collagen type III and I dominate. Type III collagen is found distributed in the interstitium of the bronchial tree, bronchial lamina propria and mainly found as a component of the alveolar wall, in intimate association with elastin. CIII is a substrate for interstitial collagenase (collagenase-1, MMP-1, expressed by monocytes, macrophages), neutrophil collagenase (collagenase-2, MMP-8, expressed by neutrophils) and neutrophil elastase (expressed by neutrophils) (3). MMP-1 has a 20 times higher preference for CIII than for CI. Transgenic mice expressing human MP-1 develop lung emphysema spontaneously and in these mice CIII located in the alveoli is specifically degraded over CI in situ as assessed by immunohistochemistry (19). MMP-1 overexpression has been observed in alveolar macrophages in emphysema tissue from COPD patients (20). Humans with genetic aberrant CIII (Ehler-Danlos syndrome) develop lung emphysema spontaneously (21, 22). Commercial assays to a CIII propeptide are available and this peptide is used as a turnover marker, correlating with both anabolic and catabolic metabolism of CIII. Increased levels of CIII have been measured in pulmonary sarcoidosis, fibrosing alveolitis, idiopathic pulmonary fibrosis, pleural fibrosis, rheumatoid arthritis and osteoarthritis.

### Prostaglandin-H2 D-Isomerase.

There were two peptides of prostaglandin-H2 D-isomerase (PTGDS, L-PGDS) in the list, one being upregulated and the other downregulated in COPD compared to non-smokers.

Lipocalin-type prostaglandin D synthase is unique in its bifunctional character as an enzyme synthesizing prostaglandin D2 (PGD2) and a secretory protein of the lipocalin super family that operates as a carrier protein for small lipophilic molecules such as retinol. L-PGDS was identified to be the same protein as β-trace, which was originally discovered in 1961 as a major protein of cerebrospinal fluid, and PGD2 biosynthesised in the local region is potent sleep modulator, anticoagulant and vasodilator.

Urinary L-PGDS increases in the early stage of kidney injury in patients with type 2 diabetes mellitus (23). Serum L-PGDS values and urinary excretion of L-PGDS are much higher in patients with essential hypertension (EHT) than those in normotensive subjects (24).

Development of an ELISA for urinary L-PGDS determination has been reported (25). LPGDS seems to be readily detected in urine and expression is regulated during number of diseases.

### Collagen Type I.

Collagen type I (CI) was represented by 11 peptides being upregulated and by 4 peptides being downregulated in COPD. CI is widely distributed in the body and the tissue harbouring the most amount of CI is bone.

CI is the dominant collagen in the lung and, like CIII, is found distributed in the interstitium of the bronchial tree, in the bronchial lamina propria and in the alveoli. CI is irregularly distributed in the alveolar cell wall, and in this location it is less prominent than CIII. Native CI is a substrate for MMP-1 (collagenase 1), MMP-8 (collgenase-2) and MMP-13 (collagenase-3) (3).

Steroid naive COPD patients display elevated serum and urine levels of CI peptides, low bone mass density (BMD) and they develop osteoporosis (26, 27) (28). COPD patients display various systemic inflammatory disease manifestations (increased CRP, fibrinogen, TNP-α, blood leukocytes) and metabolic aberrant effects such as muscle mass loss and loss of fat free mass (FFM, anorexia), which could be connected to development of bone erosion and CI release (26). In osteoporosis activated RANKL—expressing T-cells stimulate osteoblast maturation, which leads to formation of osteoclasts degrading bone and releasing CI.

Cigarette smoke extract inhibits chemotaxis and collagen gel contraction by human osteoprogenitor and osteoblast-like cells (29).

### Alpha-1-Micro Globulin.

Alpha-1-microglobulin (AMBP; Protein HC) I was represented by 3 peptides, all three being upregulated in COPD. AMBP is expressed by the liver and synthesized as a precursor protein together with bikunin, a member of the pancreatic trypsin inhibitor family. The precursor protein is proteolytically processed and free alpha-1-microglobulin is secreted. Alpha-1-microglobulin occurs in many physiological fluids including plasma, urine, and cerebrospinal fluid. It appears not only as a free monomer but also in complexes with IgA and albumin. The protein also contains covalently linked brown-yellow chromophores of unknown structure. Alpha-1-microglobulin appears to be involved in regulation of the inflammatory process (30).

The protein, as well as its IgA complex, inhibits neutrophil chemotaxis to endotoxin-activated serum.

### Fibroblast Growth Factor.

There was one peptide from fibroblast growth factor (FGF) in the list, being upregulated in COPD.

Fibroblast growth factors (FGFs) play important roles in diverse functions including morphogenesis, cellular differentiation, angiogenesis, tissue remodelling, inflammation, and oncogenesis. FGFs contain a conserved 120-amino acid FGF core domain with a common tertiary structure. FGF is a whole family of proteins that has specific roles in diseases, involved in fibroblast phenotype regulations.

FGF2 is a wide -spectrum mitogenic, angiogenic, and neurotrophic factor that is expressed at low levels in many tissues and cell types and reaches high concentrations in brain and pituitary. FGF2 has been implicated in a multitude of physiologic and pathologic processes, including limb development, angiogenesis, wound healing, and tumour growth.

### Osteopontin.

Osteopontin was represented by two peptides, both being upregulated in COPD.

Osteopontin (OPN/SPP1) is a multifunctional protein independently associated in a broad array of pathological processes. OPN is a secreted sialic acid-rich, adhesive, extracellular matrix (ECM) protein with a cell-binding sequence that interacts with several integrins. OPN is an important bone matrix protein, where it is thought to function by mediating the adhesion of osteoclasts to resorbing bone. However, studies from the past decade have identified an alternative role for OPN in regulating tissue repair and inflammation and in the cellular immune response. OPN has been shown to regulate several aspects of lung disease such as pulmonary granuloma formation, fibrosis, and malignancy (for review see (31)).

Apart from its localisation in bone, OPN is widely expressed in non-bony sites in normal human tissue and in particular at luminar epithelial surfaces (32). Baccarini-Contri et al. (33) demonstrated that osteopontin is a constitutive component of normal elastic fibres in human skin and aorta. Antibodies raised against human bone osteopontin or against osteopontin synthetic peptide (amino acids 1-10) recognized epitopes associated with the amorphous material within elastic fibres. OPN can form cross-links to other matrix proteins including type-I collagen via transglutamination (34).

### Alpha-1-Acid Glycoprotein.

Alpha-1-acid glycoprotein (AGP) was represented by two peptides, both being upregulated in COPD.

Alpha-1-acid glycoprotein (AGP) or orosomucoid (OM) is an acute phase glycoprotein (carbohydrate moiety accounts for 45% of its weight) produced by hepatocytes and to a minor extent by lymphocytes during inflammation. Normal plasma level of AGP is 0.75 mg/ml. The pro-inflammatory role of AGP is unknown but later in the immune response the glycosylation of AGP is changed, which also affects the immunomodulatory role of AGP. Changes in the sialylation and fucosylation results in expression of the sialyl Lewisx (SLEX) blood group structure on AGP. The same SLEX structure is expressed on activated leukocytes and is required for binding to E-selectin on endothelial cells and thus mediating adhesion. Both the titer (2-5 fold increase) and SLEX form of AGP is increased in established RA sera and decreases upon successful treatment. The role of AGP in established inflammation could thus be to dampen the influx of inflammatory cells to the tissue. Transcription of AGP is dependent on both IL-1, IL-6 and TNF-α activity. There are IL-1 and IL-6 responsive elements in the regulatory upstream region of the AGP gene.

### Fibrinogen.

Fibrinogen was represented by two peptides, both being upregulated in COPD

Fibrinogen is an acute phase protein that is required for the formation of fibrin clots, which are found in inflamed tissue. Fibrinogen secretion is stimulated by IL-6 and IL-6 type cytokines like oncostatin M, IL-11 and LIF which all bind the IL-6 receptor. IL-6 is under the control of TNF-α, and thus also drives the expression of fibrinogen.

Plasma fibrinogen is elevated in patients with stable COPD. Acute exacerbations of COPD are accompanied by elevations of plasma fibrinogen and serum IL-6 levels (35). Serum titres of fibrinogen are increased in RA patients and correlate well with erythrocyte sedimentation rate (ESR), an in-direct measurement of fibrinogen content of plasma, which is a clinical hallmark of RA (4). Rheumatoid arthritis (RA) patients receiving antibodies specific for TNF-o have decreased serum titres of IL-6 and fibrinogen (5).

Fibrinogen, CRP and alpha-1-antitrypsin are systemic markers of inflammation in COPD, not necessarily related to activity in the diseased tissue as such, but correlating with clinical disease activity in COPD.

### Peptide Sequences of COPD Biomarkers According to the Invention.

COPD biomarkers identified by the present inventors include those provided in Table 1 herein, SEQ ID Nos 1 to 41.

In another aspect the invention provides a diagnostic test to detect COPD or to identify the susceptibility of a patient to develop COPD which comprises measurement of one or more of the peptides listed in Table 1, SEQ ID Nos 1 to 41, or a protein comprising one or more of said peptides, in a biological sample obtained from the patient. Preferably the peptides are identified and measured using antibodies that bind to the peptides.

Antibodies used in the Identification of COPD Biomarkers.

In a further aspect the present invention provides the use of one or more antibodies listed in Table 2 in the identification of a COPD biomarker in a sample of biological fluid isolated from a subject. A person skilled in the art will recognize that antibodies against the proteins or peptides of the invention can be readily raised. Table 2 below provides a list of antibodies known to bind the biomarkers of the invention.

TABLE 2

| Protein id | Immunoassay | Source |
|---|---|---|
| Collagen type I | ELISA, Crosslaps | Nordic |
| peptide | | Biosciences, DK |
| Collagen type III | ELISA, Mab, | Abcam |
| peptide | Polyclonal Abs | Acris antibodies |
| Prostaglandin- | ELISA (reference | Santa Cruz |
| H2D-isomerase | 25), Polyclonal Abs | Cayman |
| Alpha-1- | ELISA | Alpco |
| microglobuline | | Research |
| | | Diagnostics Inc |
| Alpha-1-acid | ELISA, Poly abs | ICL labs |
| glycoprotein | | |
| Alpha-1-antitrypsin | ELISA, Poly abs | ICL labs |
| FGF basic | ELISA | Abcam |
| | | R&D |
| Osteopontin | ELISA | Bioscience |
| | | Technology |
| Fibrinogens | ELISA | Abcam |

The invention will now be described with reference to the following examples, which should in no way be considered limiting of the invention.

### EXAMPLE 1

### Materials and Methods.

### Urine Samples.

Patients with COPD were compared with asymptomatic smokers and healthy non-smokers as controls. The clinical material used in this study was collected at Gentofte University Hospital in Denmark in a strategic collaboration with Professor Asger Dirksen evaluating laboratory measurements with clinical parameters including pulmonary function, and lung structure by CT and HRCT

The subject was requested to empty his/her bladder at 18.00, then urine was collected in a 1-L plastic bottle between 18.00 and 08.00 in the morning the day after and the subject emptied his/her bladder again at 08.00. The bottle was stored at 4° C. and directly transported to AstraZeneca R&D Lund where the urine was divided into 10-mL aliquots and frozen at −80° C. In some cases the patient produced more than 1 L of urine. In those cases the total urine volume was recorded and pooled before aliquoted.

### Urine Sample Preparation.

Frozen urine (10-mL tubes) was thawed and the pH adjusted with concentrated phosphoric acid to 2.5. A 5-mL aliquot was filtered through a 0.22 μm filter and 3.0 mL injected into the LC-system. The remaining 5 mL was stored frozen.

### Instrumentation

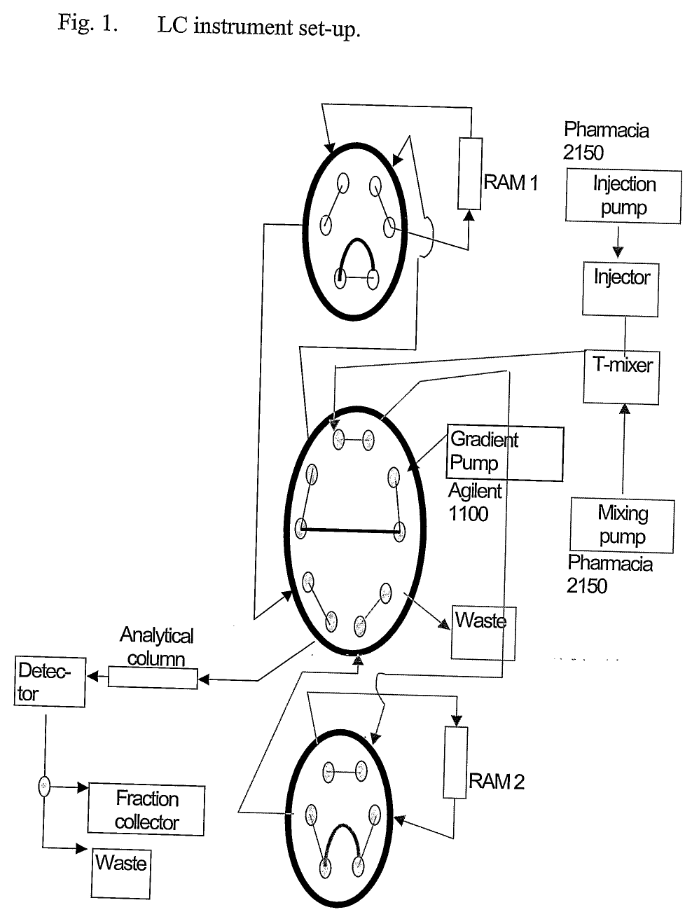

### LC Instrument Set-Up (FIG. 1)

Two Pharmacia 2150 pumps

Agilent 1100 system including gradient LC-pump, autosampler and detector (DAD)

Gilson FC 204 fraction collector

Two RAM columns: LiChrospher 60 XDS, 25 μm, (Merck), packed in NovoGROM Prep glass columns 40 mm×15 mm i.d.

One Analytical column: GROM-Sil 100 SCX, 5 μm, 50×4.6 mm

### Mobile Phases

A: Phosphate buffer 20 mM, pH 2.5, with 5% methanol

B: Phosphate buffer 20 mM, pH 2.5, 1.5 M NaCl, with 5% methanol

### Chromatographic Procedure

Urine (pH adjusted and filtered, 3.0 mL) was transferred to glass vials and placed in the Agilent 1100 autosampler where they were kept at 8° C. The autosampler loaded the urine sample into a 3-mL loop. The sample was then injected onto the RAM column #1 at a flow rate of 0.13 mL/min of mobile phase A delivered from a Pharmacia 2150 pump and simultaneously diluted 10 times with mobile phase A at a flow of 1.2 mL/min from another Pharmacia 2150 pump. After the loading was complete the Pharmacia pumps were kept running at 0.13+1.2 mL/imin to wash the RAM column until the UV response at 214 nm was back close to baseline ( ca 1.5 h). During this process RAM column #2 and the analytical SCX column were flushed by the Agilent 1100 pump (FIG. 2A).

The valves were then switched to elute the peptides from RAM column #1 by back-flushing with a gradient from the Agilent 1100 pump at a flow rate of 0.5 ml/min (gradient: 0% to 100% mobile phase B over 20 min). The peptides eluted from the RAM column were transferred directly to the analytical SCX column and separated by the gradient. The effluent was collected in 1-min fractions (500 μL per fraction) up to 45 min. After the elution step the RAM column was reconditioned by repeated quick gradients of 0% to 100% of mobile phase B. During this process a second sample was injected onto RAM column #2.

### ZipTip Desalting and Concentration of LC Fractions

In a pilot experiment fractions collected between 1 and 13 min were found never to contain any peptides. These fractions were therefore discarded before further processing. Only fractions 14-45 were analysed.

Aliquots (150 μL) of the 1-min fractions were transferred to a 96-well PCR plate (ABgene, AB-0800) and desalted and concentrated by ZipTip (μ-C18) extraction using a MassPrep sample-handling robot (Packard Multiprobe II).

The ZipTips were wetted in acetonitrile and equilibrated in 1% formic acid in water. Peptides were then bound to the ZipTip C18 material by pipetting 20 μL of a urine fraction up and down 15 times, washed by pipetting 20 μL of 1% formic acid up and down 5 times and finally eluted in another 96-well plate by pipetting 15 μL of 60% methanol in 1% formic acid up and down 4 times.

### MALDI-MS.

A 1.5-μL aliquot of the desalted and concentrated urine fractions were spotted onto a MALDI target plate (96 positions Teflon coated, Applied Biosystems) and evaporated to dryness. The 1.5-μL spotting and evaporation was repeated once followed by spotting of 0.6 μL α-cyano matrix solution. The matrix solution was prepared by diluting 1 volume of the commercial solution (α-cyano-4-hydroxycinnamic acid, Agilent) with 3 volumes of 75% acetonitrile/25% of 1% formic acid.

MALDI mass spectra were acquired on a Voyager DE-Pro instrument (Applied Biosystems) in reflector mode over a mass range of m/z 780-4000. For each spectrum 100 laser shots were accumulated on 3 different positions on the sample spot.

### Calibration and Peak Extraction

Each mass spectrum was initially calibrated using external calibration from a calibration file acquired in the middle of the target plate. The spectrum was baseline subtracted, noise filtered and deisotoped and then internally calibrated using a reference file containing urine peptide masses.

### Statistical Methods

The softwares used include Simca (Umetrics AB, Sweden) and the R packages pamr and RandomForests.

### Univariate Group Comparisons.

Wilcoxon Rank Sum test has been used in comparing pairs of the groups healthy non-smokers, asymptomatic smokers and COPD, while the Kruskal-Wallis has been used to test the overall hypothesis of no difference between groups.

### Multivariate Analysis.

The multivariate technique Partial Least Squares Discriminant Analysis (PLS-DA) has been used to build classifiers on sets of peptides to achieve good specificity and selectivity in discriminating between subject categories. Principal Component Analysis (PCA) has been used to find groups and outliers among the samples in an unsupervised manner.

Persons skilled in the art will recognise that it will be possible to use the read-outs and models from machine learning and statistical classification to define quantitative or qualitative profiles distinguishing different subject categories such as COPD and healthy never smokers.

### Summed Spectrum Analysis.

A special analysis was performed on summed spectra, where the summation of intensities was over the fractions, such that one measurement for each subject and mass was produced. Using these data two pattern recognition methods were used: Random Forests (54) and Peak Probability Contrasts (PPC) (55). The importance of each mass in the prediction of class was calculated. For Random Forests a measure of decrease in accuracy was calculated for each mass by excluding that mass from the predictive model. For PPC a measure of distance between profiles was calculated for each mass, indicating the importance of that particular mass in the prediction.

### Peptide Identification

Peptides were identified by tandem mass spectrometry (MS-MS) on an Applied Biosystems 4700 Proteomics Analyzer (MALDI-TOF-TOF configuration). The LC fractions were spotted, after desalting and concentration, on a 192 positions stainless steel plate and analysed by MALDI ionization with α-cyano as the matrix.

Tandem mass spectrometry for peptide identification was also performed using electrospray ionisation on a Waters/Nicromass Q-TOF2 instrument (ESI-quadrupole-TOF configuration). Desalted and concentrated LC fractions were directly analysed by static nanospray ESI from metal coated nanospray needles (Protana, Odense).

The MS-MS spectra were submitted for data base search using Mascot (Matrix Science).

### 3. Results

### 3.1 Clinical Characteristics of the Subjects

The study described herein includes the following subject groups: current smokers with COPD (n=20), asymptomatic smokers at risk of developing COPD (n=10) and healthy never smokers (n=10). The clinical characteristics of the subjects groups are shown in Table 3.

At Visit 1 the subject underwent physical examination and were questioned about his/her smoking history, disease history and medication use. If the subject fulfilled all inclusion and none of the exclusion criteria, then the subject entered the study. A comprehensive lung function test including dynamic and static lung volumes, diffusion capacity and a reversibility test was performed. Visit 2 included CT scans of the lungs by volume scan and HRCT. At visits 3 and 4 (two weeks after visit 3) blood, urine, induced sputum and exhaled breath condensate were collected.

The results of the lung function and spirometry testing showed that the FEV1 % of predicted, FEV1/FVC, sgaw and DLco were all reduced in smokers with COPD in comparison with never smokers and asymptomatic smokers.

Measurements of lung structure by CT and HRCT showed that the mean lung density and density of the 15th percentile were reduced in smokers with COPD compared with never smokers and asymptomatic smokers. Additionally, the relative area of emphysema was increased in smokers with COPD compared with never smokers and asymptomatic smokers. HRCT data indicated that three patients in the COPD group did not have emphysema, and these three patients were excluded before the final statistical evaluation.

TABLE 3

| | | | | | | | | | | | lung density | | |
|---|---|---|---|---|---|---|---|---|---|---|---|---|---|
| | | | | | | | | | | | Dens. | Dens. 15- | |
| | | | | | | | lung function | | | | Tot. (HU) | perc. | Rela- |
| | | Gender | | | | Pack- | | FEV1 % | | FEV1/ | neg. | point (HU) | tive |
| GROUP | n = | M//F | | Age | Weight | year | FEV1 | pred | DLCO | FVC | values | neg. val. | area |
| COPD | 20 | 11//9 | Mean | 64 | 71 | 45 | 1.62 | 57 | 4.9 | 54 | 833.5 | 912.5 | 20.6 |
| patients | | | Range | 49-80 | 48-102 | 22-69 | 0.94-2.73 | 37-76 | 2.50-9.50 | 42-67 | 764.9-873.0 | 847.6-947.9 | 0.5-44.1 |
| Healthy | 10 | 5//5 | Mean | 58 | 70 | 37 | 2.90 | 101 | 5.8 | 78 | 795.7 | 873.5 | 5.5 |
| smokers | | | Range | 47-73 | 48-92 | 28-46 | 1.95-3.22 | 85-107 | 4.20-7.90 | 73-87 | 717.1-844.7 | 807.5-909.4 | 0.4-14.5 |
| Never- | 10 | 5//5 | Mean | 58 | 80 | — | 3.13 | 104 | 8.0 | 80 | 790.2 | 873.5 | 4.7 |
| smokers | | | Range | 47-73 | 66-129 | — | 2.02-4.08 | 93-121 | 5.50-11.80 | 76-89 | 733.1-838.4 | 834.4-906.5 | 0.3-12.6 |

### Table 3 Describes the Clinical Characteristics of the Patients.

### Peptide Profiling

Peptide profiling in urine samples was performed using aliquots of the study samples. The peptide profiling data obtained by MS analysis was evaluated by applying different statistical tools and combining the results into a common score by multivariate analysis. Most of the fraction-mass values were statistically upregulated in COPD, and less than 20% of the peptides were downregulated. All peptides were submitted for sequencing and identification by mass spectrometry. For those cases where specific sequence could not be obtained, the causes were due to either too low urine concentration of the peptide, inconclusive sequence matches, or inconclusive de novo sequencing.

There were 74 peptides identified in the first pass of sequencing. Altogether these annotations could be attributed to 20 different precursor proteins. A fraction of these proteins could be grouped into a number of classes based upon their biological function:

Zinc-alpha-2-glycoprotein

Alpha-1-antitrypsin

Collagen type I

Collagen type III

Prostaglandin-H2 D-isomerase

Alpha-1-microglobulin

Fibroblast growth factor

Osteopontin

Alpha-1-acid glycoprotein

Fibrinogen

### Discussion

The peptide profiling data generated a list of peptides of potential biological interest. The peptides were selected by ranking based on several statistical parameters, obtained in comparisons between COPD patients and non-smokers.

Some of the peptides in the list could be sequenced and identified, demonstrating that some proteins were represented by multiple peptides. A short description of some of the proteins and their potential links to COPD is further provided in the detailed description of the invention.

The intensities of the majority of the urinary peptides (after normalisation to urine creatinine concentration) were found to be higher in COPD patients relative to non-smokers. Increased amounts of urinary peptides of a protein may reflect increased turnover both as a function of anabolic processes as well as degradation of the protein. In the disease pathology of COPD there is gradual degradation of matrix proteins, such as elastin, during development of emphysema. But as for other matrix degrading diseases there is also a compensatory protein synthesis, e.g. increased collagen deposition and collagen remodelling can be observed in situ in emphysemic tissue sections from COPD patients (11, 12). Depending of type and stage (early-late, high to low erosive) of emphysema pathology the tissue derived biomarkers found in different bio fluids may vary between patients (13).

### CONCLUSION

In summary, a novel 2D liquid chromatography method combined with mass spectrometry has been used to identify a number of peptide biomarkers in urine from COPD patients vs. healthy controls. The biomarkers fall into a limited number of biological effect areas, some of which may be related to known pathological processes in COPD such as lung matrix biology (e.g. collagen type I and III) and others, which may indicate novel disease mechanisms.

### REFERENCES

1. Schriver E E, Davidson J M, Sutcliffe M C, Swindell B B, Bernard G R. Comparison of elastin peptide concentrations in body fluids from healthy volunteers, smokers, and patients with chronic obstructive pulmonary disease.762-6, 1992 April.

2. Stone P J, Gottlieb D J, Oconnor G T, Ciccolella D E, Breuer R, Bryanrhadfi J, et al. Elastin and collagen degradation products in urine of smokers with and without chronic obstructive pulmonary disease. American Journal of Respiratory & Critical Care Medicine 1995;151(4):952-59.

3. Owen C A, Campbell E. The cell biology of leukocyte-mediated proteolysis. J Leukoc Biol 1999;65(2):137-50.

4. M Nishimura ea. Proteases and Chemotactic Factors in BAL Fluid From Subjects With Subclinical Emphysema. Chest 2000;117:282S.

5. Gan W Q, Man S F, Senthilselvan A, Sin D D. Association between chronic obstructive pulmonary disease and systemic inflammation: a systematic review and a meta-analysis.Thorax 2004;59(7):574-80.

6. Issaq H J, Conrads T P, Prieto D A, Tirumalai R, Veenstra T D. SELDI-TOF MS for diagnostic proteomics. Analytical Chemistry 2003;75(7):148A-55A.

7. Heine G, Raida M, Forssmann W G. Mapping of peptides and protein fragments in human urine using liquid chromatography-mass spectrometry. Journal of Chromatography A 1997;776(1):117-24.

8. Sorace J M, Zhan M. A data review and re-assessment of ovarian cancer serum proteomic profiling. BMC Bioinformatics 2003;4(24).

9. Finlay G, O'Donnell MD, O'Connor C M, Hayes J P, FitzGerald M X. Elastin and collagen remodeling in emphysema. A scanning electron microscopy study. Am X Pathol 1996;149(4):1405.

10. Lang M R, Fiaux G W, Hulmes D J, Lamb D, Miller A. Quantitative studies of human lung airspace wall in relation to collagen and elastin content. Matrix Biol 1993;13(6):471-8.

11. Cocci F, Miniati M, Monti S, Cavarra E, Gambelli F, Battolla L, et al. Urinary desmosine excretion is inversely correlated with the extent of emphysema in patients with chronic obstructive pulmonary disease. Int J Biochem Cell Biol 2002;34(6):594-604.

12. Kennedy M, Heikema A, Cooper A, Bjorkman P, Sanchez L. Hydrophobic ligand binding by Zn-alpha 2-glycoprotein, a soluble fat-depleting factor related to major histocompatibility complex proteins. Journal of Biological Chemistr 2001;276(37):35008-13.

14. Bing C, Bao Y, Jenkins J, Sanders P, Manieri M, Cinti S, et al. Zinc-alpha2-glycoprotein, a lipid mobilizing factor, is expressed in adipocytes and is up-regulated in mice with cancer cachexia. Proceedings of the National Academy of Sciences of the United States of America 2004;101(8):2500-5.

15. Ghafouri B, Stahlbom B, Tagesson C, Lindahl M. Newly identified proteins in human nasal lavage using two-dimensional gel electrophoresis and peptide mass fingerprinting.Proteomics 2002;2(1): 112-20.

16. Lindahl M, Stahlbom B, Tagesson C. Identification of a new potential airway irritation marker, palate lung nasal epithelial clone protein, in human nasal lavage fluid with twodimensional electrophoresis and matrix-asited laser desorption/ionization-time of flight. Elecrophoresis 2001;22:1795-800.

17. Stoller J K, Aboussouan L S. Alphal-antitrypsin deficiency 5: intravenous augmentation therapy: current understanding. Thorax 2004;59(8):708-12.

18. Foronjy R F, Okada Y, Cole R, J. D'Armiento. Progressive adult-onset emphysema in transgenic mice expressing human MMP-1 in the lung. Am J Physiol Lung Cell Mol Physiol 2003;284(5):L727-L37.

19. Shiomi T, Okada Y, Foronjy R, Schiltz J, Jaenish R, Krane S, et al. Emphysematous changes are caused by degradation of type III collagen in transgenic mice expressing MMP-1. Exp Lung Res 2003;29(1-15):1.

20. Cupo L N, Pyeritz R E, Olson J L, McPhee S J, Hutchins G M, McKusick V A. Ehlers-Danlos syndrome with abnormal collagen fibrils, sinus of Valsalva aneurysms, myocardial infarction, panacinar emphysema and cerebral heterotopias. Am J Med 1981;71(6):1051-58.

21. Dowton S B, Pincott S, Dernmer L. Respiratory complications of Ehlers-Danlos syndrome type IV. Clin Genet 1996;50(6):510-4.

22. Hirawa ea. Urinary Prostaglandin D synthase (beta-trace) excretion increases in the early stage of diabetes mellitus. Nephron 2001;87:321-27.

23. Hirawa ea. Lipocalin-type prostaglandin synthase in essential hypertension. Hypertension 2002;39:449-54.

24. Oda ea. Development and Evaluation of a Practical ELISA for Human Urinary Lipocalin-Type Prostaglandin D Synthase. Clinical Chemistry 2002;48(9): 1445-53.

25. Bolton C E, Ionescu A A, Shiels K M, Pettit R J, Edwards P H, Stone M D, et al. Associated Loss of Fat Free Mass and Bone Mineral Density in Chronic Obstructive Pulmonary Disease. Am J Respir Crit Care Med 2004.

26. McEvoy C E, Ensrud K E, Bender E, Genant H K, Yu W, Griffith J M, et al. Association between corticosteroid use and vertebral fractures in older men with chronic obstructive pulmonary disease. Am J Respir Crit Care Med 1998;157:704-.

27. Kochetkova E A, Gel'tser B I, Sernisotova E F, Volkova M V. Biochemical and immunological markers of osteoporosis in patients with chronic obstructive pulmonary disease. Klin Med (Mosk) 2002;80(10):31-35.

28. Liu X, Kohyama T, Kobayashi T, Abe S, Kim H J, Reed E C, et al. Cigarette smoke extract inhibits chemotaxis and collagen gel contraction mediated by human bone marrow osteoprogenitor cells and osteoblast-like cells. Osteoporosis international 2003;14(3):235.

29. Mendez E, Fernandez-Luna J L, Grubb A, Leyva-Cobian F. Human protein H C and its IgA complex are inhibitors of neutrophil chemotaxis. Proc. Nat. Acad. Sci 1986;83:1472-75.

30. O'Regan A. The role of osteopontin in lung disease. Cytokine Growth Factor Rev 2003;14(6):479-88.

31. Brown L F, Berse B, Water LVd, Papadopoulos-Sergiou A, Perruzzi C A, Manseau E J, et al. Expression and distribution of osteopontin in human tissues: widespread association with luminal epithelial surfaces. Mol Biol Cell 1992;3(10):1169-80.

32. Baccarini-Contri M, Taparelli F, IPasquali-Ronchetti. Osteopontin is a constitutive component of normal elastic fibers in human skin and aorta. Matrix Biol 1994;14:553-60.

33. Kaartinen M T, El-Maadawy S, Rasanen N H, McKee MD. Tissue transglutaminase and its substrates in bone. J Bone Miner Res 2002;17(12):2161-73.

34. Wedzicha J A, Seemungal T A, MacCallum P K, Paul E A, Donaldson G C, Bhowmik A, et al. Acute exacerbations of chronic obstructive pulmonary disease are accompanied by elevations of plasma fibrinogen and serum IL-6 levels. Thromb Haemost 2000;84(2):210-5.

35. Malmstrbm J, Larsen K, Hansson L, Lbfdahl C -G, Jensen O N, Marko-Varga G, et al. A Proteornic Feasibility Study of Central Human Pulmonary Fibrotic Tissue—Applying Nanocapillary Affinity Extraction to Improve Protein Identification. Proteomics 2002;2:394-404.

36. Chiap P, Rbeida O, Christiaens B, Hubert P, Lubda D, Boos K S, et al. Use of a novel cation-exchange restricted-access material for automated sample clean-up prior to the determination of basic drugs in plasma by liquid chromatography. Journal of Chromatography 2002;A. 975(1):145-55.

37. Breiman L. Random forests. Machine Learning 2001;45(1):5-32.

38. Tibshirani R, Hastie T, Narasimhan B, Soltys S, Shi G, Koong A, et al. Sample classification from protein mass spectroscopy by “peak probability contrasts”. Bioinformatics 2004.

39. Aebersold R and Mann M. Mass spectrometry-based proteomics, Nature 2003; 422: 198-207.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, molecular biology and biotechnology or related fields are intended to be within the scope of the following claims.

1. A COPD biomarker selected from the group consisting of the following: zinc-alpha-2-glycoprotein, alpha-1-antitrypsin, collagen type III, prostaglandin-H2 D isomerase, collagen type I, alpha-1-microglobulin, fibroblast growth factor, osteopontin, alpha-1 acid glycoprotein 2, and fibrinogen alpha-E chain.

2. A COPD biomarker, which has the amino acid sequence of any of the peptides in the group provided in Table 1.

3. A COPD disease profile, wherein that disease profile comprises the identity of two or more COPD biomarkers according to claim 1.

4. A COPD biomarker according to claim 2, wherein the COPD biomarker is selected from the group consisting of the following: zinc-alpha-2-glycoprotein, alpha-1-antitrypsin, microglobulin, fibroblast growth factor, osteopontin, alpha-1 acid glycoprotein 2, and fibrinogen alpha-E chain.

5. A method for the detection of one or more biomarkers of the disease COPD which method comprises the steps of:
(a) Obtaining a sample of biological fluid selected from the group consisting of: blood, urine, plasma, sputum, serum, saliva, cerebral-spinal fluid, sweat and tissue extracts;
(b) Subjecting the material according to step (a) to one-dimensional or multi-dimensional liquid chromatography (nD-LC);
(c) Subjecting the material according to step (b) to mass spectrometry, and
(d) Identifying the peptides of interest within the biological sample treated as described in steps (b) and (c) from the output from step (c).

6. A method according to claim 5 wherein the biological fluid is urine.

7. A method according to claim 5 wherein step (b); involves the use of cation exchange restricted access material (RAM).

8. A method according to claim 5 wherein step (b) further involves the sue of a cation exchange-column, preferably a strong-cation exchange column.

9. A method according to claim 5 wherein step (c) is performed by matrix-laser assisted laser desorption/ionization mass spectrometry (MALDI-MS).

10. The use of one or more antibodies in the group consisting of those antibodies listed in Table 2, herein in the identification of a COPD biomarker in a sample of biological fluid isolated from a subject.

11. The use according to claim 10 wherein the biological fluid is any of those in the group consisting of lung tissue, blood, urine, plasma, sputum, serum, saliva, cerebral-spinal fluid, and sweat.

12. A method for one or more of the following: predicting or diagnosing the onset or occurrence of COPD; assessing predicting or diagnosing the onset or occurrence of emphysema, which method comprises the steps of:
(a) Obtaining a sample of biological fluid from a subject to be assessed,
(b) Identifying or quantifying the peptides present within that biological fluid and using that data in order to generate a COPD disease profile, and
(c) comparing the disease profile obtained according to step (b) with that of a healthy control subject.

13. A method for assessing COPD disease progression or assessing the progression of emphysema in a subject which method comprises the steps of:
(a) preparing a COPD disease profile at a first time interval, and
(b) comparing that COPD disease profile according to step (a) with a disease profile prepared at a second time interval.

14. A method for assessing the effectiveness of one or more COPD and/or emphysema treatment regimes in a subject which method comprises the steps of:
(a) preparing a COPD disease profile at a first time, interval, and
(b) comparing that COPD disease profile according to step (a) with a disease profile prepared at a second time interval; wherein the second time interval corresponds to a time subsequent to a COPD or emphysema treatment regimen being administered to the subject.

15. A diagnostic test to detect COPD or to identity the susceptibility of a patient to develop COPD which comprises measurement of one or more of the peptides listed below, or a protein comprising one or more of said peptides, in a biological sample obtained from the patient;
YADKPETTKEQLGEF
ADKPETTKEQLGEF
MGKVVNPTQK
EDPQGDAAQKTDTSHHDQDHPTF
YVEKGTQGKIVDL
LMIEQNTKSPLF
MGKVVNPTQK
YVVHTNYDE
NGDDGEAGKPGRPGERGPPGP
GANGAPGNDGADGDAGAPGAPGSQGAPG
SPGSGPDGKTGPP
ADGQPGAKGEPGDAGAKGDAGPPGPA
GKNGDDGEAGKPGRPGERGPPGPQ
SPGPDGKTGPP
NGAPGNDGAKGDAGAPGAPGSQGAPG
EGSPGRDGSPGAKGDRGETGPA
ADGQPGAKGEPGDAGAKGDAGPPGP
DAGAPGAPGGKGDAGAPGERGPPG
GSPGSPGPDGKTGPPGP
ADGQPGAKGEPGDAGAKGDAGPPGPA
ERGEAGIPGVPGAKGEDGKDGSPGEPGANG
GAPGQNGEPGGKGERGAPGEKGEGGPPG
APGAPGGKGDAGAPGERGPPG
NDGAPGKNGERGGPGGPGP
GAPGQNGEPGGKGERGAPGEKGPGGPPG
ERGEAGIPGVPGAKGEDGKDGSPGEPGANG
GNDGAPGKNGERGGPGGPGP
DGAPGKNGERGGPGGPGP
DGAPGAPGGKGDAGAPGERGPPG
NGEPGGKGERGAPGEKGEGGPPG
KNGETGPQGPPGPTGPGGDKGDTGPPGPQG
DEAGSEADHGTHSTKRG
DEAGSEADHEGTHSTKR
LPDGSAQGT
EKQLYNKYPDA
DSRGKDSYETSQL
YSRTQTPRAEL
YRSPHWGSTY
WRQVEGMED
WRQVEGMED

16. A diagnostic test according to claim 15 wherein diagnosis of COPD or determination of susceptibility to COPD comprises measurement of an elevated level of one or more of the peptides shown in claim 15, or a protein comprising one or more of said peptides, compared with the level of said peptide or protein in patients without COPD.

17. A diagnostic test according to claim 15 wherein a protein is measured using an immunoassay.

18. A diagnostic test according to claim 15 wherein a peptide is measured using mass spectrometry.

19. A diagnostic test according to claim 15 wherein said diagnostic test is to determine susceptibility to COPD following administration of a drug to a patient.
